(19) United States
(12) Patent Application Publication
(10) Pub. No.: US 2005/0129747 A1
(43) Pub. Date: Jun. 16, 2005

(54) Use of one or more natural or modified oxygen carriers, devoid of plasma and cellular membrane constiuents, for externally treating open, in particular chronic wounds

(76) Inventors: Wolfgang Barnikol, Mainz (DE); Alexander Teslenko, Hagen (DE)

Correspondence Address: WILLIAM COLLARD;COLLARD & ROE, P.C., 1077 NORTHERN BOULEVARD, ROSLYN, NY, 11576, US

(21) Appl. No.: 10/508,092

(22) PCT Filed: Mar. 4, 2003
(86) PCT No.: PCT/EP03/02193

(30) Foreign Application Priority Data
Mar. 20, 2002 (DE) 102 12 321.7

(51) Int. Cl. A61K 38/42; A61L 15/00

(52) U.S. Cl. 424/445; 514/6

(57) ABSTRACT

The present invention relates to the use of one or more natural or modified oxygen carriers, devoid of plasma or cellular membrane constituents, for the production of an agent for the external treatment of open wounds, particularly chronic wounds. Hemoglobin or myoglobin of human or animal origin are suitable as oxygen carriers. The oxygen carriers can also preferably be modified. Suitable modifications are cross-linking, reaction with polyalkylene oxides, chemically reactive or chemically non-reactive effectors, or combinations. The agent is applied to the wound area particularly by means of spraying on an aqueous solution containing the oxygen carrier(s). The oxygen carriers can be used in particularly effective manner in the case of chronic wounds resulting from tissue degeneration, particularly diabetic tissue degeneration.

### OBJECT OF THE INVENTION

The present invention relates to the use of one or more natural or modified oxygen carriers, devoid of plasma or cellular membrane constituents, for the production of an agent for the external treatment of open, in particular chronic wounds. Hemoglobin or myoglobin of human or animal origin is particularly suitable as an oxygen carrier. The oxygen carriers can also preferably be modified. Suitable modifications are cross-linking, reaction with polyalkylene oxides, chemically reactive or chemically non-reactive effectors, or combinations. The agent is applied to the area of the wound, in particular by spraying an aqueous solution containing the oxygen carrier(s) on. The oxygen carriers can be used in particularly effective manner for chronic wounds resulting from tissue degeneration, particularly diabetic tissue degeneration.

### BACKGROUND OF THE INVENTION

Different methods are used for treating wounds, depending on the status. First, a wound that is still open must be disinfected and thereby protected against negative external influences. This can be done by means of suitable disinfectant solutions or spray-on bandages or also by applying iodine solution. Actual wound healing must then take place from the inside. This means that the blood vessels still in place must supply the destroyed tissue with sufficient amounts of substrates, so that the tissue repair mechanism can start.

Wounds can be caused by various factors, such as injuries, or also after operations or traumatic events.

On the other hand, it is known that wound formation, particularly also chronic wounds, can also be provoked by diseases, in which degeneration and/or constriction of large and/or small blood vessels occurs. This can occur, in the case of older patients, due to extended stays in bed (decubitus). Another example of this is diabetes mellitus—so-called blood sugar disease—which results in demonstrable degeneration and arteriosclerosis (P. Carpenter, A. Franco, Atlas der Kapillaroskopie [Atlas of Capillaroscopy], 1983, Abbott, Max-Planck-Inst. 2, D-Wiesbaden) of the large and small blood vessels (macroangiopathy and microangiopathy of the arteries). Here, it was furthermore possible to determine a reduction in this variable, particularly of the skin surrounding the wound, by means of measuring the so-called transcutaneous oxygen partial pressure. This means that an oxygen deficiency (hypoxia) is present here. 40 mmHg is considered to be a critical value (C. D. Müller et al., Hartmann Wund [Wound] Forum 1 (1999), 17-25).

The blood flows to the tissues, including the skin, through the arteries. It constantly supplies the cells with substrates required for life. Any degeneration of these blood vessels results in a deficient supply of substrates to the cells, and to their death. The substrates must overcome the last, seemingly insignificant path of approximately 20 μm from the smallest blood vessels (capillaries) to the cells by means of diffusion or filtration; in this connection, oxygen plays a special role, because the organism has particular difficulty in handling this substrate.

There are three problems involved here:
(1) It is true that oxygen is absolutely essential for life (a human being is brain-dead after only approximately five minutes if his/her brain does not receive oxygen), but at the same time, oxygen is highly toxic (a newborn that receives respiration treatment with pure oxygen will die-after only a few days).
(2) Oxygen has very little solubility in an aqueous medium. This has the result, according to FICK's first law, of a lesser diffusivity of oxygen. In addition, there is a fundamental law of diffusion, namely SMOLUCHOWSKI's and EINSTEIN's law, that says that the diffusion speed (of oxygen) decreases with an increasing diffusion distance. Now the diffusion constant of oxygen is so small that at a diffusion distance of as little as 20 μm, the diffusion speed is only 5% of the initial value. Therefore a water layer of only 50 μm represents practically complete oxygen insulation for the cells. Oxygen is footsore, so to speak. It is transported along the long paths in the organism from the lungs to the tips of the toes convectively with the bloodstream, bound to hemoglobin, and only in this way is able to overcome the long distances in a manner that is practical for the organism.
(3) For oxygen, in contrast to glucose, for example, there is no storage area in the body, therefore this substrate must be available to the cells at all times and quickly, in a sufficient amount; oxygen is a so-called immediate substrate that is necessary for life.

The organism has solved these problems using several mechanisms. The toxic effects of oxygen are avoided in that the latter binds to hemoglobin and thereby remains harmless. At the same time, the free oxygen is diluted and thereby further loses its harmful oxidative potential. Nevertheless, it is instantaneously available in a sufficient amount, because the bond with hemoglobin is reversible. The problem of the low diffusive range is solved in that the organism has developed a very finely dispersed blood vessel network (capillary network), which ensures that on the average, every cell is at a distance of at most 25 μm from a capillary; in this way, the diffusion path of oxygen in the organism remains below the critical length of 50 μm. In addition, a cell can be diffusively supplied with oxygen from several sides; this represents a safety mechanism. The immediate availability, in keeping with the demand (oxygen is not allowed to be available in excess, otherwise it would have a harmful effect) is achieved, in the organism, by means of vascular regulation of the blood vessel flow, which controls perfusion and thereby optimizes the supply of oxygen.

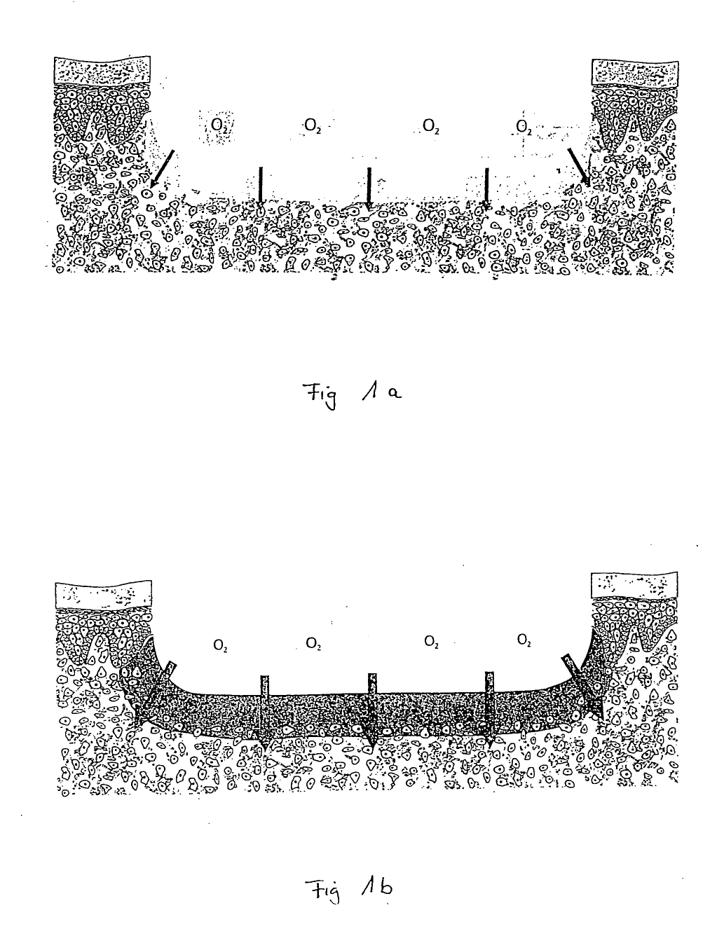

If there is an open wound surface, the diffusive oxygen supply to the surface cell layer, from many sides, is eliminated. This cell layer is like a cell culture. Its oxygen supply from the outside is poor because an aqueous liquid film forms above the cell layer, which film, as explained, forms a diffusive oxygen barriers in accordance with the laws of diffusion. This is illustrated by the following FIG. 1a, in which the water layer that forms above the cells of the wound floor is indicated schematically. Fresh wounds in normal tissue can heal in a few days, in the most advantageous case, if the oxygen supply from underneath, in other words from the inside, is sufficient. However, it was possible to show, in animal experiments, that such fresh wounds heal even better if the oxygen concentration of the surrounding air is increased (M. P. Pai et al., Sug. Gyn. Obstet. 135 (1972), 756-758). Older, particularly chronic wounds, cannot be simulated in animal experiments. In humans, however, they are known to heal very slowly, or not at all, because of their marked oxygen deficiency.

In order to now be able to heal chronic wounds better, as well, so-called hyperbaric oxygen therapy (HBO) has been used. In this treatment, patients are placed in pressurized chambers, where they are subjected to an excess pressure of pure oxygen of about 3 bar for a certain period of time, about one hour, in so-called passes. Normal wound therapy comprises approximately 40 such passes (C. D. Müller et al., Hartmann Wund Forum 1 (1999), 17-25). In fact, wound healing is achieved in this manner. However, multiple treatments prove to be less successful, and the effect also decreases with the number of passes. This can be explained: While the oxygen supply to the surface wounds is increased, this is achieved at the cost of a toxic effect of the concentrated oxygen at high pressure, as explained above; in the final analysis, the harmful effect presumably predominates.

The U.S. Pat. No. 2,527,210 from the year 1944 describes a hemoglobin solution that can allegedly be used for the treatment of wounds, both intravenously and topically, for example by spraying it on. In this connection, the hemoglobin is obtained from fresh erythrocytes that are subjected to freezing shock after centrifugation and drawing off the blood plasma fraction. This results in cell lysis, and hemoglobin is released. The broken-up cell walls are also present in the product. This formulation is a concentrated cell detritus (cell fragments). In this way, an antiseptic cover effect such as otherwise achieved with iodine solution, after having added 5% sodium sulfide, is supposed to be achieved for a surface treatment. In other words, the wound is merely closed here. In order to correctly adjust the viscosity of the product, for example for use as a spray, plasma is added. Oxygen transport is not mentioned here.

This path of use of such hemoglobin products was obviously left behind during subsequent times. Thus, WO 97/15313 describes the therapeutic use of hemoglobin for improving wound healing. For this purpose, hemoglobin free of stroma and pyrogens is systemically administered to the patients, in other words intravenously, particularly after operations and traumatic events, in order to increase the blood pressure. In particular, a hemoglobin cross-linked with diaspirin is used for this purpose.

Systemic, intravenous administration of hemoglobin can, however, exert only the known, one-sided, indirect effect on wound healing, since supply to the blood vessels located in the wound must take place from the inside, and therefore no possibility of treatment from the outside, and also no possibility of overcoming the diffusion barrier described above exists.

Furthermore, application of cellular membrane constituents onto open wounds is questionable, since it is known that some of the phospholipids that flow out of the cell membranes are highly toxic. In a message published in the Internet on Mar. 14, 2002 (www.sangui.de/en/Stock/news: study demonstrates effectiveness of oxygen in skin treatment), it is reported that emulsions containing natural or synthetic oxygen carriers can be used to treat aging skin or wrinkles. However, this does not involve open wounds, in which a barrier layer is present, as has been explained.

Task of the Invention

It is therefore the task of the present invention to use such a product for the treatment of open wounds, with which oxygen is transported from the outside, into the blood vessels in question, particularly the damaged blood vessels, in local, targeted manner, without intervening in the organism as a whole, in other words systemically; this makes it possible to avoid adverse side effects.

Explanation of the Invention

This task is accomplished, according to the invention, in that an agent is made available, which contains a natural or synthetic oxygen binder (or carrier) devoid of plasma and cellular membrane constituents, the latter produced from a natural one by means of suitable modification, or mixtures thereof, and that this agent is introduced into the aqueous oxygen barrier layer of the wound floor. In this connection, because of the oxygen binders (carriers) that are introduced, an effective oxygen transport through the barrier layer occurs, because of the mechanism of facilitated diffusion (see FIG. 1b).

Surprisingly, what happens here is not that the wound is covered and closed. Furthermore, accelerated wound healing is possible by means of supplying oxygen to the blood vessels that are no longer intact, from the outside. By means of this mechanisms, the oxygen is offered to the cells of the wound floor in physiological manner, namely from the bond with hemoglobin, in non-toxic form, and in a sufficient amount, directly at the desired location, and oxidative damage is avoided.

This is particularly surprising because the state of the art teaches that external wound healing is more likely with antiseptic agents such as iodine or hydrogels (cf. WO 97/15313, page 3, paragraph 1), or that treatment from the inside out is required, and that an external use of hemoglobin results in sealing of the wound, i.e. that particularly effective wound healing is supposed to be achieved by means of external occlusion with hypotoxia, cf. Gretenar et al., Schweiz. Med. Forum, 2001, 237-242.

According to the invention, a natural (native) oxygen carrier, particularly hemoglobin or myoglobin or a modified derivative thereof, or mixtures thereof, is/are used. The modification can be intramolecular cross-linking, polymerization (intermolecular cross-linking), pegylation (covalent linking with polyalkylene oxides), modification with chemically reactive effectors such as pyridoxal-5′-phosphate or 2-nor-2-formyl-pyridoxal-5′-phosphate, or also with chemically non-reactive effectors of the oxygen bond, such as 2,3-bisphosphoglycerate, inositol hexaphosphate, inositol hexasulfate, or mellitic acid, or a combination thereof. For myoglobin, intramolecular cross-linking is not possible, as it is for hemoglobin, but all other modifications are possible. Such products are known and described, for example, in DE-A 100 31 744, DE-A 100 31 742, and DE-A 100 31 740. Cross-linking of oxygen carriers is also described in DE 197 01 37, EP 97 1000790, DE 44 18 937, DE 38 41 105, DE 37 14 351, DE 35 76 651. These known methods are therefore incorporated here.

Particularly preferred modified oxygen carriers are hemoglobins having a molecular weight of 65,000 to 15,000,000, such as intramolecularly cross-linked products such as those according to WO 97/15313, particularly polymer products as well as intermolecularly cross-linked products having an average molecular weight of 80,000 to 10,000,000 g/mol, particularly 100,000 to 5,000,000, or analogously produced myoglobins having a molecular weight of 16,000 to 5,000,000, particularly 100,000 to 3,000,000, preferably 1,000,000 g/mol. Those oxygen carriers that are polymerized, for example using cross-linking agents known for intermolecular modification, such as bifunctional cross-linking agents like butadiene diepoxy, divinyl sulfone, diisocyanate, particularly hexamethylene diisocyanate, cyclohexyl diisocyanate, and 2,5-bisisocyanatobenzol sulfonic acid, di-N-hydroxy succinimidyl ester, diimidoester, or dialdehyde, particularly glyoxal, glycol aldehyde that reacts analogously, or glutardialdehyde are particularly preferred.

Furthermore, those products that are polymerized in this manner and pegylated with a polyethylene glycol or suitable derivative thereof are preferred. This includes, for example, polyethylene oxide, polypropylene oxide, or a copolymer of ethylene oxide and propylene oxide, or an ester, ether, or ester amide thereof. It is furthermore preferred if the covalently linked polyalkylene oxide has a molar mass of 200 to 5000 g/mol.

For covalent linking of the polyalkylene oxides, those derivatives of polyalkylene oxide that contain a linking agent already covalently bound with a functional group, thereby allowing a direct chemical reaction with amino, alcohol, or sulfhydryl groups of the hemoglobins, forming covalent links of the polyalkylene oxides, are preferably used, for example polyalkylene oxides with reactive N-hydroxy succinimidyl ester, epoxy (glycidyl ether), ldehyde, isocyanate, vinyl sulfone, iodacetamide, imidazolyl formate, tresylate groups, and others. Many such monofunctionally activated polyethylene glycols are commercially available.

The production of such modified oxygen carriers is described in the German patent applications cited above, and incorporated herein.

Modified cross-linked (intramolecular or intermolecular), or cross-linked and pegylated hemoglobin products having an average molecular weight of 250,000 to 750,000 g/mol, or myoglobin products having an average molecular weight of 50,000 to 750,000 g/mol, are very particularly preferred. Above all, those products that are additionally modified with chemically reactive or chemically non-reactive effectors of the oxygen bond, or a combination thereof, are preferred.

In this connection, the oxygen binder, which also acts as an oxygen carrier, can have a human or animal origin, such as an equine, bovine, or preferably porcine origin. In this connection, the product is purified to be devoid of plasma and cellular membrane constituents, by means of suitable known measures such as centrifugation and fractionated ultrafiltration. Cell lysis by means of deep freezing does not take place, since otherwise the desired composition cannot be obtained. The product is furthermore free of stroma and pyrogens.

In particular, the oxygen carriers produced in this manner can also be purified as described, for example by means of chromatography (e.g. by means of preparative volume exclusion chromatography), by means of centrifugation, filtration, or ultrafiltration, separated into fractions of different molecular weights, and subsequently processed further, cf. for example DE-A 100 31 740 or WO 02/00230.

Human or porcine hemoglobin, which is natural or modified as described, is particularly preferred as an oxygen carrier.

In addition, myoglobin can also be used. In this connection, natural human myoglobin is preferred, but any other myoglobin of animal origin, or also myoglobin modified as described, is possible. This is obtained as described above for hemoglobin, but no intramolar cross-linking is possible.

Mixtures of natural and modified oxygen carrier can also be used, such as, for example, in a ratio of 20:1 to 1:20, with reference to weight.

Mixtures of hemoglobin and myoglobin, or their modified derivatives, are also possible, in the aforementioned ratio of 20:1 to 1:20.

The agent is made available by means of introduction of the oxygen carrier into an aqueous medium, as described below.

According to the invention, the treatment of open wounds, particularly chronic (in other words no longer fresh) wounds takes place in humans and in animals, whereby use in humans is preferred, by means of topical treatment with the oxygen carriers described. The oxygen carrier(s) is/are dissolved in an aqueous medium, in an amount of 0.1 to 35 wt.-%, particularly 0.1 to 20, above all 0.1 to 15 wt.-%, in order to apply them. The carrier, i.e. the aqueous medium can, in particular, also have physiologically compatible electrolytes, such as salts, in suitable amounts. These include sodium chloride, potassium-calcium-magnesium chloride, sodium hydrogen (bi)carbonate, sodium citrate, sodium lactate. These are preferably present in a physiological concentration or also a multiple thereof e.g. 10 times, but also in amounts of 0.1 to 30 wt.-%, whereby sodium chloride is particularly preferred for this. The electrolytes can also be present in a mixture.

If necessary, other additives can be present, namely 0 to 20, preferably 0.1 to 20, particularly 0 to 15 wt.-% preferably 0.1 to 15, particularly 0.1 to 10 wt.-%. These are particularly nutrients for cells. They are particularly selected from among glucoses, e.g. in amounts of 0.1 to 5 wt.-%, insulin in amounts of up to 25 IU/ml, the natural amino acids known for the application in question, in other words amino acids known for humans or for the animals in question, e.g. 0 or 0.1 to 5 wt.-%, or also tissue factors, such as interleukins in physiological amounts, up to a 10-fold amount thereof.

If necessary, it can be advantageous if antioxidants, such as acetyl cysteine, superoxide dismutase, in amount of 0.001 wt.-% to 2 wt.-%, are furthermore contained as additives. In this case, if hemoglobin/a derivative is used as the oxygen carrier, the latter will also act as a katalase.

The agent is applied externally. Depending on the state of the wound, it is rubbed in or, preferably, sprayed on in a fine spray. In this connection, one or several different oxygen carriers can be used. For example, the natural oxygen binder(s) can be used in the agent in a particularly high concentration, the modified product(s) also in a particularly low concentration, as needed, or also, a combination of both groups can be selected, if: the viscosity is to be particularly adjusted for spray application. Otherwise, the selection of the oxygen binder(s) and its/their concentration is independent, in each instance, and equally effective.

According to the invention, it has been shown that open wounds, particularly also chronic wounds having very different causes, can be effectively treated. These can be wounds after operations, after trauma, after injuries, or also wounds caused by degenerative changes in the tissue. In this connection, they can be wounds caused by generative changes of the arterial blood vessels and wounds resulting from chronic venous insufficiency. These particularly include decubitus as well as chronic wounds, particularly those resulting from diabetes.

### EXAMPLES

In the following, the invention will be explained in greater detail, using the following examples.

I. Production of Agents According to the Invention

### Example 1

Human natural hemoglobin was freed from plasma and cellular membrane constituents by means of centrifugation and ultrafiltration, and purified.

Of this, 8 wt.-%, as well as 5 wt.-% glucose and 20 IU/ml insulin, were dissolved in 100 ml water, containing 0.9 wt.-% sodium chloride.

### Example 2

Highly pure porcine hemoglobin, in a concentration of 330 g/L, dissolved in an electrolyte having the composition 50 mM NaHCO₃ and 100 mM NaCl, was deoxygenated at 4° C. by stirring the solution while constantly renewing the pure nitrogen atmosphere above the solution. Subsequently, 4 mol sodium ascorbate (as a 1 molar solution in water) was added per mol (monomer) hemoglobin, and this was allowed to react for 6 h. The solution was titrated to a pH of 7.1 with 0.5 molar lactic acid, 1.1 mol pyridoxal-5′-phosphate per mol hemoglobin was added, and this was allowed to react for 16 h. Now a pH of 7.8 was adjusted with 0.5 molar soda lye, 1.1 mol sodium borhydride (as a 1 molar solution in 0.01 molar soda lye) was added, and this was allowed to react for one hour. Now a pH of 7.3 was adjusted with 0.5 molar lactic acid, then 1.1 mol 2,3-bisphosphoglycerate per mol hemoglobin and, after 15 min reaction time, 8 mol glutardialdehyde per mol hemoglobin, dissolved in 1.8 L pure water, was added within 5 minutes, and allowed to react for 2.5 h. After titration with 0.5 molar soda lye to a pH of 7.8, 15 mol sodium borhydride (as a 1 molar solution in 0.01 molar soda lye) was added per mol hemoglobin, for 1 h. This was followed by an addition of 2 liters water per liter of original hemoglobin solution. The pH was then 9.3, and an addition of 4 mol methoxy-succinimidyl propionate polyethylene glycol, having a molecular weight of 2000 g/mol, took place for 2 h. The nitrogen atmosphere above the solution was replaced with pure oxygen.

After 1 h, insoluble constituents were removed by means of centrifugation (20,000 g for 15 min). Subsequently, there was a change in the electrolyte, by means of volume exclusion chromatography (Sephadex G-25 gel, Pharmacia, Germany) to produce an aqueous electrolyte solution having the composition 125 mM NaCl, 4.5 mM KCl, and 20 mM NaHCO₃.

The yield was 77%; the yield for molecular weight greater than 700,000 g/mol is 28%.

Measurements of the characteristics of the oxygen bond under physiological conditions (a temperature of 37° C., a carbon dioxide partial pressure of 40 Torr, and a pH of 7.4) resulted in a p50 value of 22 Torr and an n50 value of 1.95 for the product. This oxygen carrier is particularly suitable for use according to the invention, in aqueous solution, as described in Example 1.

### Example 3

Synthesis of human hemoglobin cross-linked with glutardialdehyde took place as in Example 2, but using highly pure, concentrated human hemoglobin and using a 16 times molar excess of the cross-linking agent. Polymers were obtained by means of fractionation of the solution of the cross-linking products using preparative volume exclusion chromatography (in accordance with EP-A 95 10 72 80.0: “Verfahren zur Herstellung molekular-einheitlicher hyperpolymerer Hämoglobine” [Method for the production of molecular-uniform hyperpolymer hemoglobins] with Sephacryl S-300 HR gel, Pharmacia Biotech, Freiburg, Germany) (here, as the first eluted 57 mass-% of the cross-linked hemoglobin).

The cross-linked hemoglobins were divided into two parts, A and B. The hemoglobin A (compare FIG. 3) proved to be predominantly polymer hemoglobin having a modal value of the molecular weight distribution of 950 kg/mol (compare Example 1). Covalent binding of monofunctionally active mPEG-SPA-1000 took place analogous to the method of procedure described in Example 2 for cross-linked porcine hemoglobin. After the addition of sodium hydrogen carbonate (up to 150 mM) to the solution of the polymers, it was possible for a 12 times molar excess mPEG-SPA-1000 to react with the hemoglobin monomers. Subsequent to a reaction time of one hour, lysine was added in a 60 times molar excess, to “catch” any active molecules of the mPEG-SPA-1000. Both the cross-linked hemoglobin according to Solution A and the cross-linked and pegylated product according to Solution B are suitable for use according to the invention.

### Example 4

Cross-linked bovine hemoglobin was produced by means of cross-linking highly pure, concentrated bovine hemoglobin with a 14 times molar excess of glutardialdehyde, in accordance with Example 2, molecular fractionation of the synthesis products, binding of mPEG-SPA-1000 in accordance with Example 2 and 3, respectively. A molecular weight distribution of the non-modified hemoglobin polymer is shown in FIG. 5, namely an eluogram of a volume exclusion chromatography (using the gel “Sephacryl S-400 HR,” Pharmacia Biotech, Freiburg, Germany); here, the modal value of the molecular weight distribution is 810 kg/mol.

### Example 5

Highly pure, concentrated, deoxygenated porcine hemoglobin, dissolved in an aqueous electrolyte having the composition 50 mmol/L NaHCO₃ and 100 mmol/L NaCl was reacted at room temperature with a 14 times molar excess of glutardialdehyde. Sodium cyanoborhydride, added to the (monomer) hemoglobin in a 10 times molar excess, reduced the Schiff's bases that were formed during cross-linking, and stabilized the covalent cross-linking. The solution of cross-linked hemoglobins that was obtained was divided into three parts (A, B, and C), and processed further in different ways.

Part A remained unchanged, the determination of the molecular weight distribution (according to Pötzschke, H., et al. (1996, Macromolecular Chemistry and Physics 197, 1419-1437, as well as Pötzschke, H., et al. (1996, Macromolecular Chemistry and Physics 197, 3229-3250), using volume exclusion chromatography with the gel Sephacryl S-400 HR (Pharmacia Biotech, Freiburg, Germany), resulted in a modal value of the molecular weight distribution of 520 kg/mol for the cross-linked porcine hemoglobin.

The polymers of Part B were covalently linked with monofunctionally active mPEG-SPA-1000 (Shearwater Polymers Europe, Enschede, Netherlands): First, sodium hydrogen carbonate was added to the solution of the cross-linked hemoglobins as a solid substance, subsequently the addition of mPEG-SPA-1000 took place in a 12 times molar excess (with reference to the hemoglobin monomers), also as a solid substance. After a reaction time of one hour, lysine was added in a 60 times molar excess (with reference to hemoglobin), and reacted with any remaining active mPEG-SPA-1000 molecules.

Part C: The same method of procedure as described for Part B was carried out with the solution of the cross-linked hemoglobins, but using mPEG-SPA-2000 (Shearwater Polymers Europe, Enschede, Netherlands).

Subsequently, a solvent exchange took place in the three solutions A, B, and C (using ultrafiltration, “Ultraminisette 10 kDa,” Pall Gelman Sciences, Rossdorf, Germany, or volume exclusion chromatography using the gel “Sephadex G-15 M,” Pharmacia Biotech, Freiburg, Germany), to produce a solution in an aqueous electrolyte (standard solution) having the composition: 125 mM NaCl, 4.5 mM KCl, and 3 mM NaN₃.

All of the products according to solution A, B, or C are suitable for use according to the invention.

### Example 6

Intramolecularly cross-linked hemoglobin was produced as described in Example 2, but in a 0.1% concentration.

### Example 7

Commercially available natural human myoglobin (e.g. from Sigma, Germany) was purified by means of gel chromatography. This can be used according to the invention, as is or also modified as described above.

### Example 8

10% of a non-modified human hemoglobin as described in Example 1 and 5 wt.-% of a modified product as described in Example 2 were put into 100 ml purified water containing 0.9 wt.-% sodium chloride, 0.2 wt.-% sodium bicarbonate, 1 wt.-% glucose. The solution is immediately ready for use.

### Example 9

8 wt.-% of a human myoglobin modified with polyethylene glycol, produced in accordance with Example 3, Solution A, was put into 100 ml purified water containing 0.9 wt.-% sodium chloride as well as 5 wt.-% glucose, 20 IU/ml.

The solution is immediately ready for use, and particularly also stores well.

II. Examples of Use

### Example 10

A solution according to Example 2 was applied to a chronic wound on the inside of the left ankle of a female patient that had been in existence for months, due to chronic venous insufficiency, over the entire area of the wound, in a thin layer, using a fine spray. This was done twice daily. After 20 days, there was clear granulation of the wound floor, with contouring of the wound edge and formation of a temporary epithelium. The wound was closed after 2 months.

### Example 11

In the case of a male-patient, there had been an amputation wound, 10 cm long and 4 cm wide, for one year-after arterial occlusion of the left leg and removal of the forefoot (fish mouth). Treatment was performed after prior cleaning of the chronic wound with maggots and concentrated urea solution. After 4 months, the wound was closed.

1. Use of one or more natural or modified oxygen carriers, devoid of plasma or cellular membrane constituents, for the production of an agent for the external treatment of open wounds.

2. Use according to claim 1, wherein the oxygen carrier(s) is/are hemoglobin or myoglobin of human or animal origin, or modified derivatives thereof, or mixtures thereof.

3. Use according to claim 1, wherein the oxygen carrier(s) is/are selected from among natural or modified human or porcine hemoglobin or mixtures thereof.

4. Use according to claim 1, wherein modified or natural myoglobin or mixtures thereof are used.

5. Use according to claim 1, wherein hemoglobin and myoglobin or modified derivatives thereof are used in a mixture ratio of 1:20 to 20:1.

6. Use according to claim 1, wherein the modification of the oxygen carrier(s) is intramolecular, intermolecular cross-linking, pegylation, reaction with chemically reactive or chemically non-reactive effectors, or a combination thereof.

7. Use according to claim 6, wherein the modification is intermolecular cross-linking, pegylation, or a combination thereof.

8. Use according to claim 7, wherein a reaction with a chemically non-reactive or a chemically reactive effector or a combination thereof is present as the modification.

9. Use according to claim 1, wherein the oxygen carrier(s) is/are applied to the wound in the form of a solution.

10. Use according to claim 9, wherein the application of the oxygen carrier(s) takes place by means of spraying.

11. Use according to claim 1, wherein the oxygen carrier(s) is/are applied in the form of an aqueous solution containing physiologically compatible salts as well as 0.1 to 15 wt.-% of the oxygen carrier(s), and 0 to 20 wt.-% additives, i.e. in a physiological concentration or up to 10 times physiological concentration.

12. Use according to claim 11, wherein as physiologically compatible salts, those selected from among sodium chloride, sodium hydrogen carbonate, sodium bicarbonate, potassium chloride, calcium magnesium chloride, sodium citrate, sodium lactate, or mixtures thereof are contained.

13. Use according to claim 1, wherein the additives are selected from among glucose, insulin, amino acids, antioxidants, tissue factors.

14. Use according to claim 1, wherein chronic wounds, operation wounds, injury wounds, wounds after trauma are treated.

15. Use according to claim 14, wherein chronic wounds resulting from degeneration or constriction of the arterial blood vessels are treated.

16. Use according to claim 14, wherein chronic wounds resulting from diabetes are treated.

17. Use according to claim 14, wherein decubitus wounds are treated.

18. Use according to claim 14, wherein wounds resulting from chronic venous insufficiency are treated.
